(19) United States
(12) Patent Application Publication
(10) Pub. No.: US 2005/0053633 A1
(43) Pub. Date: Mar. 10, 2005

(54) Lead for writing, sketching, and cosmetics

(75) Inventors: Harald Von Godin, Oberasbach (DE); Reiner Appel, Oberasbach (DE); Gerhard Lugert, Nurnberg (DE)

Correspondence Address: LERNER AND GREENBERG, PA, P O BOX 2480, HOLLYWOOD, FL, 33022-2480, US

(73) Assignee: Faber-Castell AG

(21) Appl. No.: 10/836,148

(22) Filed: Apr. 30, 2004

(30) Foreign Application Priority Data
Sep. 9, 2003 (DE) 203 13 963.1
Sep. 23, 2003 (DE) 203 14 685.9

(51) Int. Cl. A61K 6/00; C09D 13/00

(52) U.S. Cl. 424/401; 106/31.11

(57) ABSTRACT

A lead is to be used for writing, sketching, and cosmetics. The lead is prepared by extrusion of a lead starting mass including at least one sugar and/or sugar alcohol and at most 2% water at an elevated temperature, which brings about softening of the sugar and/or sugar alcohol.

### BACKGROUND OF THE INVENTION

### FIELD OF THE INVENTION

The invention relates to a lead or chalk for writing, sketching and cosmetic purposes. Binder systems of conventional leads include cellulose derivatives such as sodium carboxymethylcellulose (NaCMC), hydroxyethyl-cellulose (HEC), methylhydroxyethylcellulose (MHEC), methylhydroxypropylcellulose (MHPC), or sodium carboxy-methylstarch (CMS). The mode of action of such binders is based on their ability to swell in water. Conventional starting masses for leads are therefore admixed with relatively large amounts of water. However, the addition of water has disadvantages: a water-containing preparation including, for example, fillers, color pigments and additives, can, due to microbiological restrictions, only be stored for several hours or a few days, particularly when it is to be used to produce cosmetic leads. A further disadvantage is that, to shape the leads, a constant moisture content within the mass must be maintained to ensure constant diameters and even lead surfaces. A further disadvantage is the obligatory drying of such leads at about 40-140° C. This not only requires increased energy expenditure, but also complex process control. Firstly, shrinkage associated with the removal of water must be taken into consideration. Secondly, the leads flexible in the moist state have to be dried at elevated temperature, thus involving energy expenditure, in order to retain the “straightness” in cases under rotational movements. In the case of so-called post-wax leads, the leads are dipped after drying into molten waxes or oils in an additional process step.

### SUMMARY OF THE INVENTION

It is accordingly an object of the invention to provide a lead for writing, sketching, and cosmetics that overcomes the hereinafore-mentioned disadvantages of the heretofore-known devices of this general type.

With the foregoing and other objects in view, there is provided, in accordance with the invention, a lead for writing, sketching, and cosmetics. The lead is made by a process that includes the step of thermoplastic processing of a lead starting mass including at least one sugar or sugar alcohol and at most 2% water at an elevated temperature to soften the at least one sugar or sugar alcohol.

According to the invention, the lead is produced by thermoplastic processing, for example in an injection molding process or preferably by extrusion of a lead starting mass that includes at least one sugar and/or sugar alcohol in solid form at elevated temperature, which brings about melting of the sugar and/or sugar alcohol. A softening and/or melting of the sugar or optionally also of other constituents can naturally not be achieved in aqueous solution. The lead starting mass is therefore anhydrous or includes at most 2% water. The invention is based on the idea of using substances, in particular near-natural ones, which melt during the shaping of the lead, for example by extrusion of the lead mass, and binding the other lead constituents. In this process, simple cooling of the lead mass gives the finished lead, meaning that complex removal of water or other solvents is unnecessary. The lead starting mixture and the lead can be stored as required for several weeks or months, with microbiological impairment barely being an issue due to the fact they do not contain water.

In a particularly preferred variant, the binder system of the lead is composed of at least one sugar and/or sugar alcohol and a cellulose derivative and/or starch derivative. Surprisingly, it has been found that even such a mixture is thermoplastically softenable and can be processed in an extrusion process. In addition, it has been found that by using a binder system of this type it is possible to produce leads that have increased breaking strength compared with leads that exclusively use sugars and/or sugar alcohols as thermoplastically softenable binder. The leads produced thermoplastically according to the invention are, moreover, distinguished by a very even, homogeneous transfer to paper. Compared with conventional leads, dusting or crumbling of the lead mass during application to paper barely arises.

Melting of sugars with celluloses and starches or derivatives thereof is often only possible with the application of increased pressure since sugars, such as glucose, fructose, sucrose, mannose etc., and also cellulose derivatives and starch derivatives have decomposition phenomena between 100 and 220° C. Homogeneous melting and/or mixing is easier when using sugar alcohols, e.g. sorbitol, mannitol, xylitol, adonitol, (penta)erythritol, arabitol, dulcitol, threitol, which can melt initially to be water-clear above 100° C. without the application of increased pressure and surprisingly take up celluloses or starches or derivatives thereof without problems. Particularly suitable cellulose derivatives are sodium carboxymethylcellulose (NaCMC) and sodium carboxymethylstarch (CMS). Intimate combining is possible here in high-speed mixers or extruders at temperatures of 80-120° C. Intimate mixing or combining can also be promoted by adding 5%-30% water to celluloses and/or starches and sugars/sugar alcohols during the mixing or compounding process, the water present being evaporated to a residual content of less than or equal to 2% during the mixing of the starting materials, for example during the compound preparation in the extruder, so that melting of the sugar or sugar alcohol is then possible and leads with a correspondingly low moisture content can be extruded.

A mass that is particularly suitable for lead production includes a binder system that is formed from 0.8% to 18%, preferably 0.8% to 8%, cellulose derivative and/or starch derivative and 4% to 40%, preferably 4% to 30% sugar and/or sugar alcohol.

The spectrum of properties for the lead, for example its marking behavior, is influenced by adding fillers in a content of from 40% to 70% and fat-like or wax-like substances in a content of from 5% to 25%. Leads are obtained that correspond approximately to the known post-wax leads, although an additional process step (dipping the leads into molten wax or oil) is dispensed with. Suitable fillers are primarily kaolin, talc, mica and titanium dioxide.

Through the use of fat-like or wax-like substances which have both lipophilic and also hydrophilic properties, for example the Na salt of a fatty acid, e.g. sodium stearate, it is possible to produce water-soluble and/or water-swellable leads.

For colored leads or chalks for the writing and cosmetics sectors, the following base formulations may be given:

Leads containing a sugar alcohol as the sole thermoplastically softenable binder:

| | |
|---|---|
| 4 to 30% | Sugars and/or sugar alcohols |
| 5 to 25% | Fat-like or wax-like substances |
| 40 to 70% | Fillers |
| Remainder: | as required, e.g. additives such as aromas, |
| | lubricants, colorants, waxes, fats, and |
| | emulsifiers. |

Lead containing a mixture of cellulose/starch derivatives and sugars/sugar alcohols:

| | |
|---|---|
| 0.5 to 18% | Cellulose derivatives and/or starch derivatives |
| 4 to 40% | Sugars and/or sugar alcohols |
| 40 to 70% | Fillers |
| Remainder: | as required, e.g. additives such as aromas, |
| | lubricants, colorants, waxes, fats and |
| | emulsifiers. |

Other features that are considered as characteristic for the invention are set forth in the appended claims.

Although the invention is illustrated and described herein as embodied in a lead for writing, sketching and cosmetics, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying examples.

### DESCRIPTION OF THE PREFERRED EMBODIMENTS

### EXAMPLE 1

Aquarellable, green colored leads, diameter 3.0 mm.

| | |
|---|---|
| 6.0% | Mannitol |
| 3.0% | Sorbitol |
| 2.0% | NaCMC (Walocel CRT 100, Wolff, Walsrode) |
| 10.0% | Sodium stearate |
| 8.0% | Ethoxy (80) cetostearyl alcohol |
| | (=Emulsifier, CAS 68439-49-6) |
| 64.0% | Kaolin |
| 7.0% | Pigment Green 7 (C.I. 74260) |

The raw materials are homogeneously mixed in a high-speed mixer between 80 and 100° C. to give a compound. Besides a dry homogenization, it is possible, to accelerate the process, to initially add a water content of from 15 to 20%, and to evaporate it again to a residual moisture of max. 2% during the mixing operation in the high-speed mixer. The resulting fine granulate can be stored over several weeks and can be shaped as required on a screw extruder (preferably 2-screw extruder) at about 150° C. to give leads. The resulting thermoplastically shaped leads exhibit good aquarellability due to the emulsifier+sodium stearate addition which is present.

### EXAMPLE 2

A comparison lead that has been produced without the addition of sodium carboxycellulose in the manner described above:

| | |
|---|---|
| 6.0% | Mannitol |
| 3.0% | Sorbitol |
| 10.0% | Sodium stearate |
| 8.0% | Ethoxy (80) cetostearyl alcohol |
| | (CAS 68439-49-6) |
| 66.0% | Kaolin |
| 7.0% | Pigment Green 7 (C.I. 74260) |

The mass can likewise be extruded to give leads, but has a transverse breaking strength that is about 25% below the transverse breaking strength of the leads according to Example 1. This shows that the higher strength of the leads according to Example 1 is the result of the interplay between sugars, in particular sugar alcohols, with cellulose ethers, e.g. sodium carboxymethylcellulose.

### EXAMPLE 3

Describes a non-aquarellable round chalk, diameter 10 mm.

| | |
|---|---|
| 12.0% | Mannitol |
| 4.0% | Pentaerythritol |
| 5.0% | Sodium carboxymethylstarch (CMS) |
| 66.6% | Talc |
| 6.0% | Fatty alcohol + approx. 50 mol/kg of ethylene oxide |
| | (Genapol T 500 powder, manufacturer: Clariant) |
| 6.4% | Pigment Yellow 3 (C.I. 11710) |

The yellow chalks are extruded. Following compound preparation in the high-speed mixer at approx. 90° C., extrusion of the chalks takes place at a temperature between 140° C. and 160° C. on a two-screw extruder.

### EXAMPLE 4

Water-resistant blue lead, diameter 4.0 mm

| | |
|---|---|
| 9.0% | Mannitol |
| 4.0% | Xylitol |
| 3.0% | Binder mixture consisting of CMC and CMS |
| | (=Crayon B 300 P, supplier: Albon-Chemie) |
| 5.0% | Sodium stearate |
| 67.0% | Kaolin |
| 2.9% | Stearic acid |
| 5.1% | Paraffin wax |
| 4.0% | Pigment Blue 15:1 (C.I. 74160) |

Following compound preparation on a two-screw extruder with a temperature control between 85 and 100° C., lead extrusion takes place at 130-150° C. The extensive water resistance is caused by the addition of wax and fatty acid.

All of the percentages are percentages by weight.

This application claims the priority, under 35 U.S.C. § 119, of German utility model application No. 203 13 963.1, filed Sep. 9, 2003, and of German utility model No. 203 14 685.9, filed Sep. 23, 2003; the disclosures of the prior applications are herewith incorporated by reference in their entirety.

1. A lead for writing, sketching, and cosmetics, comprising:
a lead starting mass including at least one sugar or sugar alcohol and at most 2% water, said lead starting mass being a thermoplastic at an elevated temperature that softens the at least one sugar or sugar alcohol.

2. The lead according to claim 1, wherein said at least one sugar or sugar alcohol includes a sugar alcohol selected from the group consisting of mannitol, sorbitol, pentaerythritol, xylitol, adonitol, arabitol, dulcitol, and threitol.

3. The lead according to claim 1, wherein said at least one sugar or sugar alcohol includes xylose.

4. The lead according to claim 1, further comprising at least one cellulose derivative or starch derivative.

5. The lead according to claim 4, wherein said at least one cellulose derivative or starch derivative includes sodium carboxymethylcellulose.

6. The lead according to claim 4, wherein said at least one cellulose derivative or starch derivative includes sodium carboxymethylstarch.

7. The lead according to claim 4, wherein:
said at least one cellulose derivative or starch derivative forms from 0.5 to 18 weight percent; and
said at least one sugar or sugar alcohol forms from 4 to 40 weight percent.

8. The lead according to claim 7, wherein:
said at least one cellulose derivative or starch derivative forms from 0.5 to 8 weight percent; and
said sugar or sugar alcohol forms from 4 to 30 weight percent.

9. The lead according to claim 1, further comprising a filler forming from 40 to 70 weight percent.

10. The lead according to claim 9, wherein said filler is selected from the group consisting of talc, kaolin, mica, and titanium dioxide.

11. The lead according to claim 1, further comprising a fat-like or wax-like substance.

12. The lead according to claim 11, wherein said fat-like or wax-like substance is selected from the group consisting of a fatty acid and a fatty acid derivative.

13. The lead according to claim 11, wherein said fat-like or wax-like substance forms from 5 to 25 weight percent.

14. The lead according to claim 1, wherein said lead starting mass is softenable into a thermoplastic when mechanically processed.

15. The lead according to claim 1, wherein said lead starting mass is softenable into a thermoplastic when extruded.
